(19) United States
(12) Patent Application Publication
(10) Pub. No.: US 2026/0294276 A1
(43) Pub. Date: Oct. 1, 2026

(54) SOMNOGRAPHY SYSTEM

(71) Applicant: Oxlantic Medical AB, Uppsala (SE)

(72) Inventors: Anders ECKERBOM, Bromma (SE); Joakim ÖSTBLOM, Uppsala (SE)

(21) Appl. No.: 19/491,002

(22) PCT Filed: Jun. 10, 2024
(86) PCT No.: PCT/SE2024/050559

§ 371 Date: Dec. 8, 2025

(30) Foreign Application Priority Data
Jun. 20, 2023 (SE) 2350750-2

(51) Int. Cl. A61B 5/097; A61B 5/083

(52) U.S. Cl. CPC A61B 5/097; A61B 5/0836; A61B 2560/0285; A61B 2562/0247

(57) ABSTRACT

A somnography system includes a capnography arrangement including an airway adapter, a pressure measurement adapter and a mainstream capnography sensor, and a portable monitor. The somnography system allow mainstream capnography measurements in connection with respiration monitoring of a user and is portable and easy to use.

### TECHNICAL FIELD

The present invention generally relates to a somnography system, and in particular to an airway adapter, an airway adapter arrangement, and a capnography arrangement that can be used in a mainstream capnography system.

### BACKGROUND

Nocturnal, laboratory-based polysomnography (PSG), also known as a sleep study, is the most commonly used test in the diagnosis of obstructive sleep apnea syndrome (OSAS), but also other sleep disorders, such as periodic limb movement disorder, narcolepsy, chronic insomnia, and rapid eye movement (REM) sleep behavior disorder. PSG is non-invasive and consists of a simultaneous recording of multiple physiologic parameters related to sleep and wakefulness. Home-based, limited-channel sleep studies are being used more often to diagnosis obstructive sleep apnea, but they have some limitations. Such sleep disease monitoring is typically done at medical facilities due to the lack of equipment enabling a home-based sleep monitoring. In particular, equipment for respiratory monitoring have traditionally been bulky and not easily operated by the user and thereby mainly been limited to laboratory-based PSG. Examples of such equipment for respiratory monitoring include capnographs for monitoring the concentration or partial pressure of carbon dioxide (CO₂) in the respiratory gases. Its main development has been as a monitoring tool for use during anesthesia and intensive care. It is usually presented as a graph of CO₂, measured in kilopascals (kPa) or millimeters of mercury (mmHg), plotted against time. Measurements taken at the end of the exhalation, are commonly referred to as end tidal CO₂ (ETCO₂).

Capnographs, i.e., CO₂ gas analyzers with a waveform display, are traditionally classified as so-called mainstream or sidestream capnographs. The fundamental difference between mainstream and sidestream capnographs is whether they divert gas from the airway for analysis. Sidestream capnographs take a small sample flow from the respiratory circuit, nostrils and/or mouth of a human subject to an adjacent instrument, in which the actual gas analysis take place, whereas mainstream capnographs measures the CO₂ concentration directly in the respiratory circuit.

Mainstream capnographs comprise a CO₂ sensor fitted to an airway adapter connected to the respiratory circuit of a human subject. The gas to be analyzed by the CO₂ sensor is, thus, taken directly from the airway. This technology generally provides very accurate readings since the CO₂ sensor is at the actual airway.

U.S. Pat. Nos. 5,464,982, 7,455,644, 7,445,602 and 8,915,861 disclose airway adapters for mainstream capnographs.

Sidestream capnographs pull a fraction of the human subject's exhaled air through a small tube from the respiratory circuit into a monitor comprising a CO₂ sensor. The readings obtained by sidestream capnographs are generally regarded as being less accurate than mainstream capnographs because of the variables involved with pulling the gas sample from the patient to the monitor, and into the internal CO₂ sensor.

US 2019/0298960, U.S. Pat. Nos. 5,857,461, 5,335,656 and 7,383,839 disclose oral nasal cannula or airway adapters that can be used for sidestream capnographs.

There is still a need for a somonography system that can used in sleep studies and monitoring at home and that includes a mainstream capnograph that is portable and could be used at the home of patients as a part of such a somnography system

### SUMMARY

It is a general objective to provide an airway adapter, an airway adapter arrangement, and a capnography arrangement that can be used in a somnography system.

This and other objectives are met by embodiments as disclosed herein.

The present invention is defined in the independent claims. Further embodiments of the invention are defined in the dependent claims.

An aspect of the invention relates to an airway adapter adapted to be disposed below nostrils of a human subject. The airway adapter comprises a prong case comprising at least one nasal prong arranged to collect nasally exhaled breath from the human subject, a pressure case in fluid communication with the prong case via a pressure channel, a pressure sensing tube in fluid communication with the pressure case and an airway case defining an airway passage in fluid communication with the prong case via a flow restriction. The airway case comprises a first light window into the airway passage and a second light window into the airway passage. The airway adapter is connectable to a mainstream capnography sensor comprising at least one light source and at least one light detector to position the mainstream capnography sensor onto the airway adapter to align the at last one light source with the first light window and align the at least one light detector with the second light window.

Another aspect of the invention relates to an airway adapter arrangement comprising an airway adapter according to above and a pressure measurement adapter attached to the pressure sensing tube. The pressure measurement adapter comprises a pressure sensing channel in fluid communication with the pressure sensing tube, a first pressure measuring port in fluid communication with the pressure sensing channel and a second pressure measuring port in fluid communication with ambient air.

A further aspect of the invention relates to a capnography arrangement comprising an airway adapter according to above or an airway adapter arrangement according to above and a mainstream capnography sensor. The mainstream capnography sensor comprises a sensor housing comprising a first light window, a second light window, at least one light source arranged in the sensor housing to emit infrared (IR) light through the first light window and at least one light detector arranged in the sensor housing to detect IR light through the second light window. The mainstream capnography sensor also comprises a sensor cable attached to the sensor housing and in electrical communication with the at least one light source and the at least one light detector. The first light window of the sensor housing is aligned with the first light window of the airway case and the second light window of the sensor housing is aligned with the second light window of the airway case when the mainstream capnography sensor is connected to the airway adapter.

Yet another aspect of the invention relates to a somnography system comprising a capnography arrangement according to above and comprising an airway adapter arrangement according to above. The somnography system also comprises a portable monitor for monitoring a spontaneously breathing human subject. The portable monitor comprises a sensor port connectable to the sensor cable of the mainstream capnography sensor, an adapter receptacle connectable to the pressure measurement adapter, a first pressure port configured to be in fluid communication with the first pressure measuring port of the pressure measurement adapter, a second pressure port configured to be in fluid communication with the second pressure measuring port of the pressure measurement adapter and a differential pressure sensor in fluid communication with the first pressure port and the second pressure port and configured to measure a pressure difference between the first pressure port and the second pressure port and generate an output signal representative of the pressure difference. The portable monitor also comprises a processor communicatively connected to the sensor port and the differential pressure sensor and a memory coupled to the processor and comprising instructions executable by the processor to cause the processor to process an output signal received at the sensor port and generated by the mainstream capnography sensor to generate a CO₂ parameter value representative of partial pressure of CO₂ in the exhaled air from the human subject and process the output signal from the differential pressure sensor to generate a respiration signal.

The present invention defines a somnography system and components thereof that can be used for sleep monitoring and studying, such as at home of a human subject. The somnography system is designed to be easy to use and handle, even by non-medically trained persons. The somnography system is further portable and can thereby be used by users at home or in ambulatory care.

### BRIEF DESCRIPTION OF THE DRAWINGS

The embodiments, together with further objects and advantages thereof, may best be understood by making reference to the following description taken together with the accompanying drawings, in which:

FIG. 1 is an illustration of an airway adapter according to an embodiment in a front view;

FIG. 2 is an illustration of the airway adapter in FIG. 1 in a rear view;

FIG. 3 is a cross-sectional view of an airway adapter according to an embodiment;

FIG. 4 is an illustration of an airway adapter and a mainstream capnography sensor according to an embodiment;

FIG. 5 is an illustration of a mainstream capnography sensor according to an embodiment;

FIG. 6 is a cross-sectional view of the mainstream capnography sensor in FIG. 5;

FIG. 7 is an overview of a somnography system according to an embodiment;

FIG. 8 is an illustration of a pressure measurement adapter according to an embodiment;

FIG. 9 schematically illustrates attachment of a pressure measurement adapter to an adapter receptacle of a portable monitor according to an embodiment;

FIG. 10 is a block diagram of a portable monitor according to an embodiment.;

FIG. 11 is an illustration of an airway adapter according to another embodiment in a front view;

FIG. 12 is an illustration of the airway adapter in FIG. 11 in a rear view;

FIG. 13 is an illustration of an airway adapter and a mainstream capnography sensor according to another embodiment; and

FIG. 14 is an illustration of an airway adapter and a mainstream capnography sensor arranged attached to the face of a human subject.

### DETAILED DESCRIPTION

The present invention generally relates to a somnography system, and in particular to an airway adapter, an airway adapter arrangement, and a capnography arrangement that can be used in a mainstream capnography system.

The present invention relates to a somnography system designed to be used not only in medical facilities but also being sufficiently small to be portable to use in ambulatory care and also by patients themselves at their homes. The somnography system has been designed to include components that are connectable and function together to enable mainstream capnography measurement together with respiratory monitoring of the user. Thus, the somnography system will not only enable accurate measurement of carbon dioxide (CO₂) in the exhalation air of a user but may also be used to monitor the respiration of the user during sleep.

The somnography system of the invention is thereby focusing on the respiratory parameters during a sleep study. PSG typically includes monitoring of other parameters than such respiratory parameters including, but not limited to, brain activity (electroencephalography (EEC)), eye movements (electrooculography (EOG), muscle activity (electromyography (EMG), and heart rhythm (electrocardiograma system of the invention is therefore denoted somnography system rather than PSG system since it monitors respiratory parameters. However, the somnography system of the invention could be used as a part of a PSG system, which then also comprises equipment for performing, for instance, EEC, EOG, EMG and/or ECG monitoring.

The somnography system comprises an airway adapter designed to be connectable to a mainstream capnography sensor forming a combined unit that can be attached on or to the face of the patient where the airway adapter will collect exhaled breaths from the patient. The collected exhaled breath is analyzed by the mainstream capnography sensor electrically connected to a portable monitor. The portable monitor is also connectable to a pressure measurement adapter arranged in fluid connection with the airway adapter to enable respiratory monitoring.

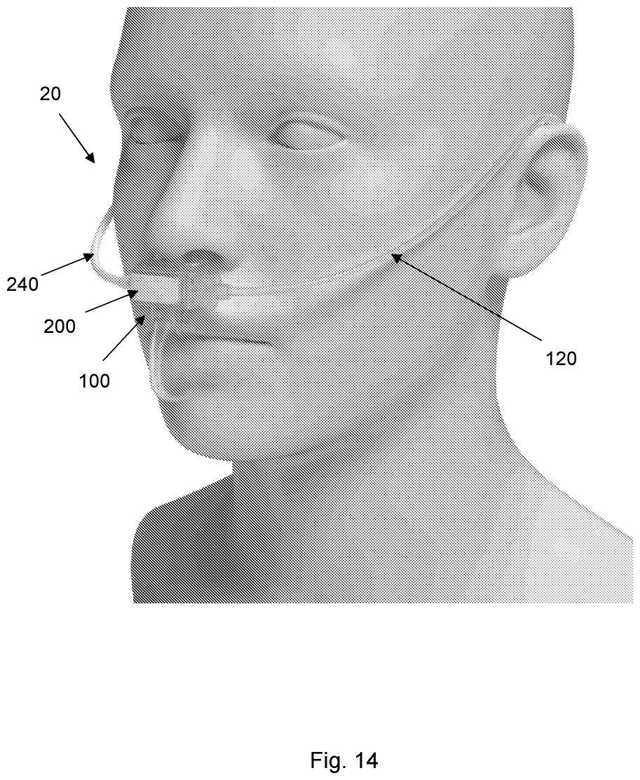

The unique design of the airway adapter and the mainstream capnography sensor and the attachment of these two units together allows an easy attachment of the airway adapter with the mainstream capnography sensor at the face of the patient without the need for dedicated attachment equipment and units by utilizing a pressure sensing tube of the airway adapter and a sensor cable of the mainstream capnography sensor as attachment equipment, see FIG. 14. This not only maintains the airway adapter attached on the face of the patient to collect exhaled air but enables this with a minimum of tubing and cables.

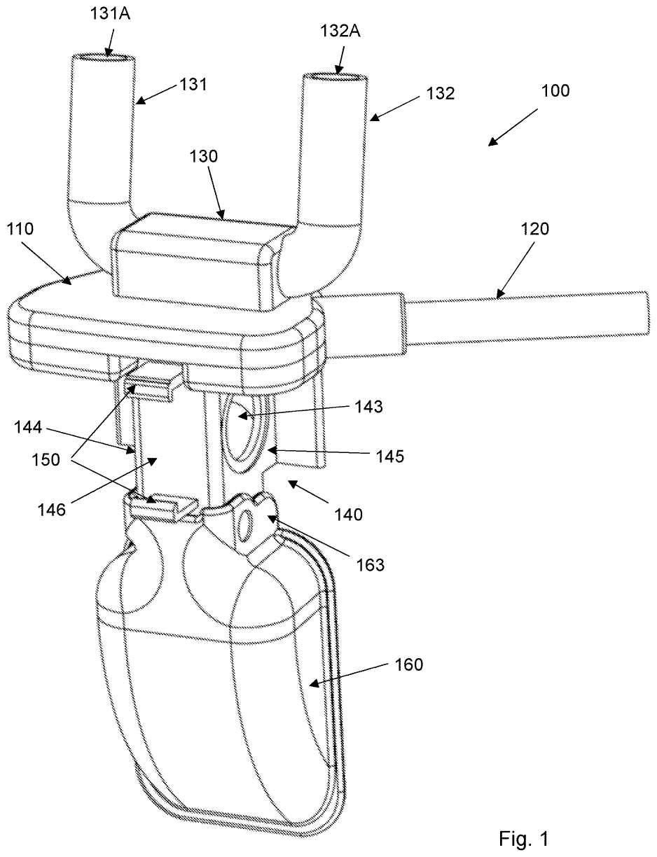

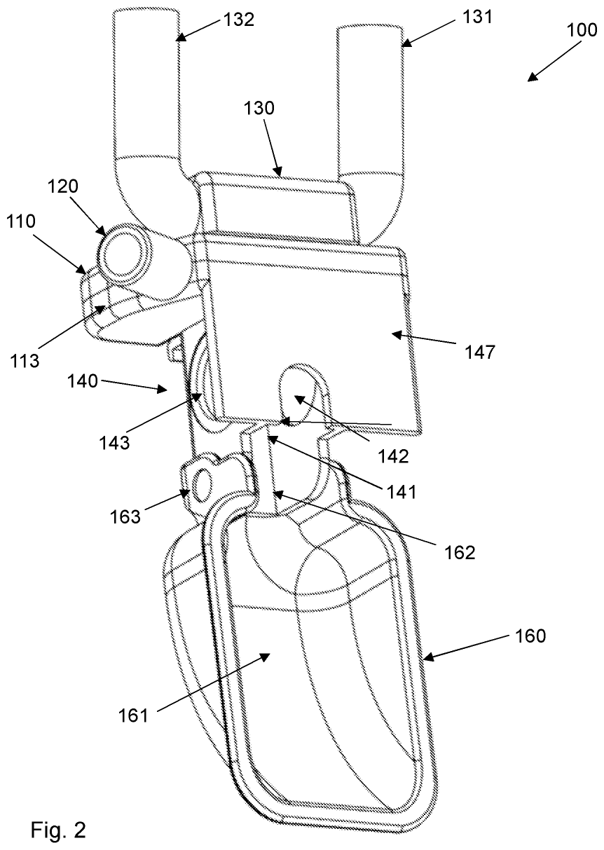

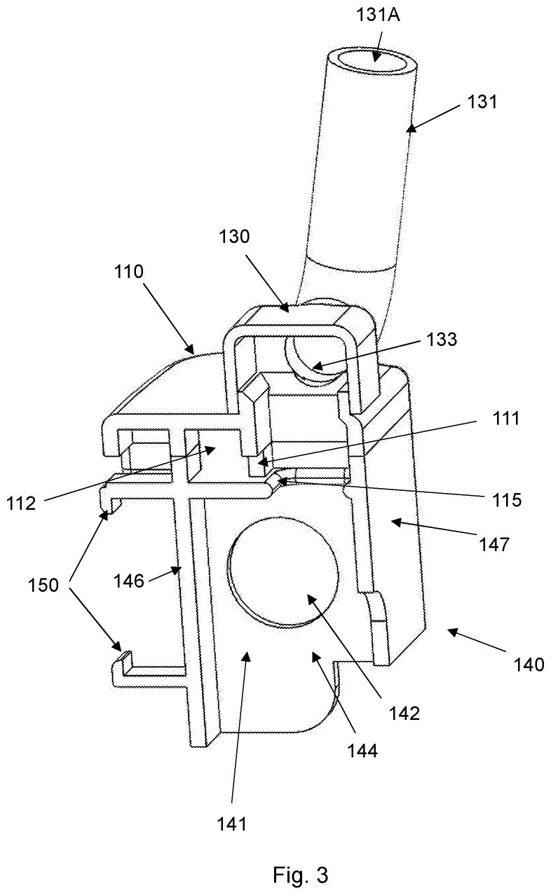

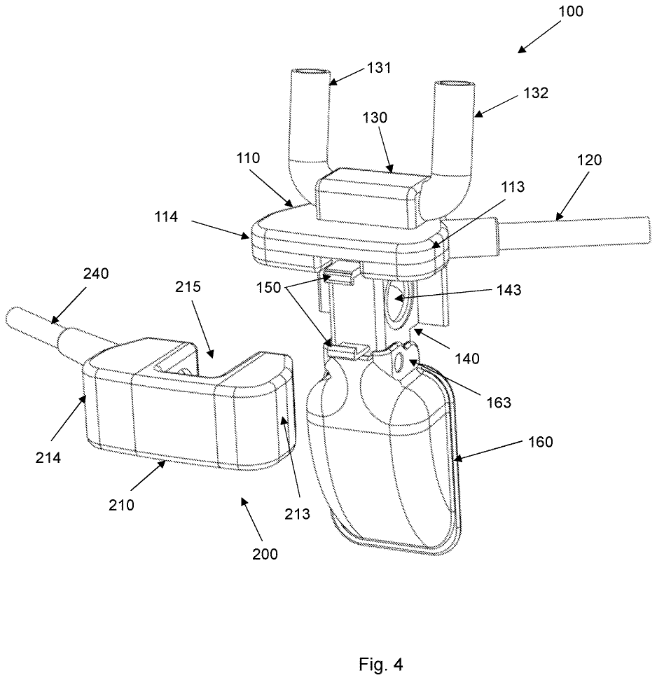

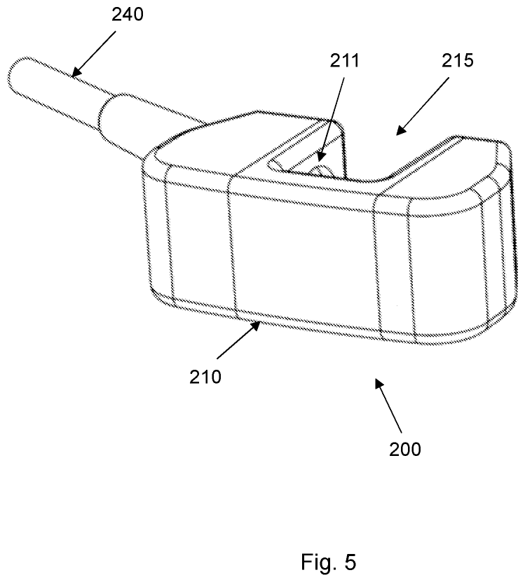

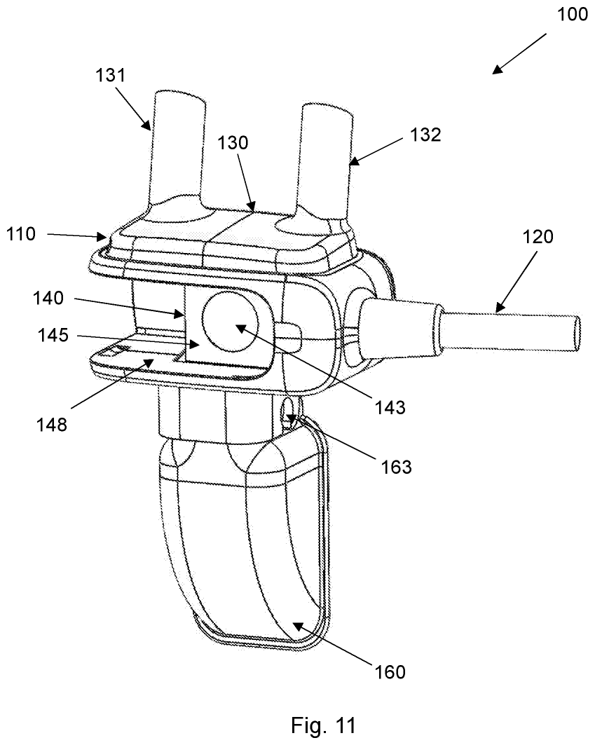

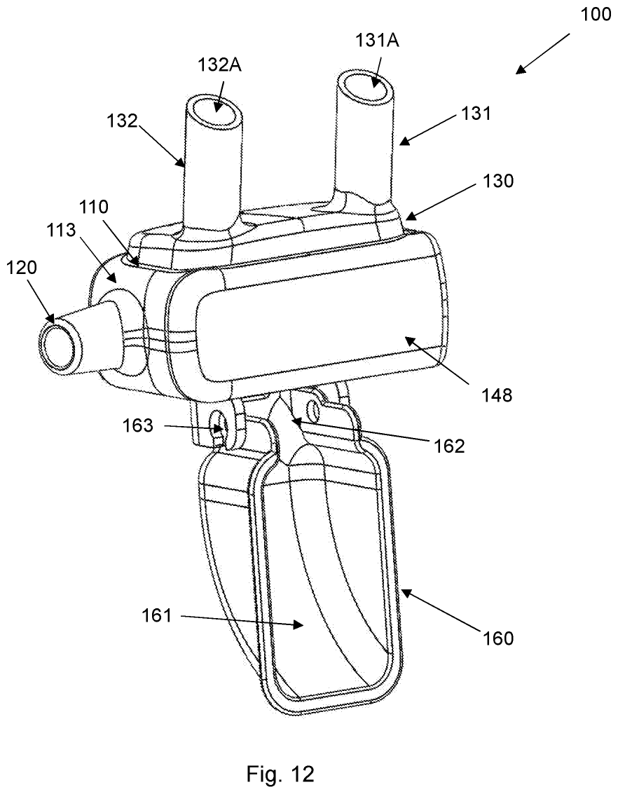

FIGS. 1, 2, 11 and 12 illustrate an embodiment of the airway adapter 100 adapted to be disposed below nostrils of a human subject in a front view (FIGS. 1 and 11) and a rear view (FIGS. 2 and 12). The airway adapter 100 comprises a prong case 130, also referred to as prong housing herein, comprising at least one nasal prong 131, 132 arranged to collect nasally exhaled breath from the human subject. The airway adapter 100 also comprises a pressure case 110, also referred to as pressure housing herein, in fluid communication, i.e., fluid connection, with the prong case 130 via a pressure channel 111, see FIG. 3. The airway adapter 100 also comprises a pressure sensing tube 120 in fluid communication with the pressure case 110. The airway adapter 100 further comprises an airway case 140, also referred to as airway housing. The airway case 140 defines an airway passage 141 in fluid communication with the prong case 130 via a flow restriction 115, see FIG. 3. The airway case 140 comprises a first light window 142 into the airway passage 141 and a second light window 143 into the airway passage 141. The airway adapter 100 is connectable to a mainstream capnography sensor 200 comprising at least one light source 220 and at least one light detector 230 to position the mainstream capnography sensor 200 onto the airway adapter 100 to align the at least one light source 220 with the first light window 142 and align the at least one light detector 230 with the second light window 143, see FIGS. 4-6, 13.

Fluid communication as used herein means that a fluid, and in particular a gas, can flow between two units or devices that are in fluid communication with each other. For instance, the prong case 130 is in fluid communication with the airway passage 141 to thereby enable gas flowing through the prong case 130 to flow into the airway passage 141. Hence, in a particular embodiment, fluid communication as used herein refers to enabling gas to flow between the units or devices. Gas as used herein include typically mean exhaled gases from a user.

The airway adapter 100 thereby comprises three main cases or housings, the pressure case 110, the prong case 130 and the airway case 140. The prong case 130 comprises at least one nasal prong 131, 132 arranged to collect nasally exhaled breath from the human subject. In an embodiment, the prong case 130 could comprise a single nasal prong arranged to collect nasally exhaled breath from the left nostril or the right nostril. However, in a preferred embodiment, the prong case 130 comprises two nasal prongs 131, 132 as shown in FIGS. 1, 2, 11 and 12, one for each nostril, i.e., a first nasal prong 131 and a second nasal prong 132. The one or two prongs 131, 132 could be in the form of tube(s) extending from the prong case 130 and designed to be at least partly inserted into the nostril(s) or end shortly below the nostrils to thereby enable collection of the nasally exhaled air. Each prong 131, 132 thereby comprises a respective channel 131A, 132A in fluid connection with a prong chamber 133 defined by the prong case 130, see FIGS. 3, 12. This means that once the human subject exhales the exhaled air will flow at least partly into the channel(s) 131A, 132A of the nasal prong(s) 131, 132 and flow into the prong chamber 133. The prong case 130 is in fluid communication with the airway case 140 or more correctly the prong chamber 133 of the prong case 130 is in fluid communication with the airway passage 141 defined by the airway case 140 via a flow restriction 115.

This means that the collected nasally exhaled air will flow through the nasal prong(s) 131, 132, into the prong chamber 133 and then through the flow restriction 115 and into airway passage 141. The flow restriction 115 as arranged between the prong chamber 133 and the airway passage 141 causes formation of an overpressure in the prong chamber 133 as the user exhales. This overpressure is propagated through pressure channel 111 separating the prong case 130 and the pressure case 110 or more correctly separating the prong chamber 133 of the prong case 130 and a pressure chamber 112 of the pressure case 110. The pressure case 110 is in fluid communication with a pressure sensing tube 120. In a typical embodiment, the pressure sensing tube 120 is connected to and ends at a side wall 113 of the pressure case 110 as shown in FIGS. 1, 2, 11 and 12. This means that the overpressure, also referred to as pressure wave or signal herein, is propagated through the pressure channel 111 into the pressure chamber 112 and further into the pressure sensing tube 120. Hence, each time the user exhales a local overpressure is built up in the prong chamber 133 due to the flow restriction 115 between the prong chamber 133 and the airway passage 141. The overpressure in turn causes the propagation of a pressure wave or signal through the pressure channel 111, the pressure chamber 112 and into and along the pressure sensing tube 120 to thereby be detectable by a portable monitor as is further described herein.

The exhaled air from the user will also flow through the flow restriction 115 and into the airway passage 141 to thereby flow past the two light windows 142, 143 in the airway case 140. The mainstream capnography sensor 200 can then analyze the exhaled air flowing through the airway passage 141 as the human subject is exhaling. This is possible by arranging the at least one light source 220 and the at least light detector 230 of the mainstream capnography sensor 200 in vicinity of and aligned with the light windows 142, 143. Hence, light from the at least one light source 220 is thereby directed through the first light window 142 into the airway passage 141 and through the second light window 143 where the light is detected by the at least one light detector 230, which is further described herein. This means that the two light windows 142, 143 and the portion of the airway passage 141 between the light windows 142, 143 form a gas cuvette, through which exhaled air samples can be analyzed by the mainstream capnography sensor 200.

The first and second light windows 142, 143 could be windows made of optically transparent material enabling the light from the light source 220 to pass through the light windows 142, 143. Illustrative, but non-limiting, examples of such materials include optically transparent plastics, glass and sapphire crystal.

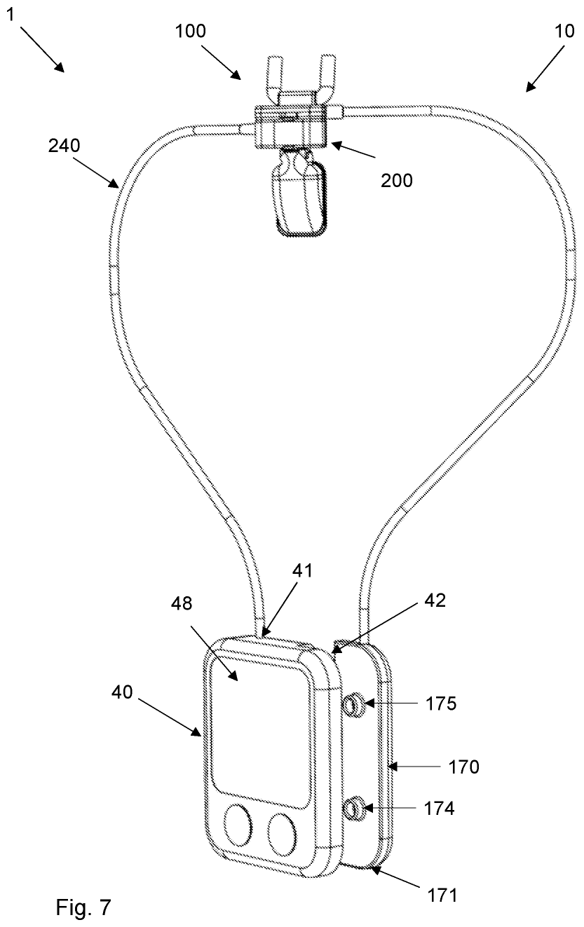

The pressure sensing tube 120 extending from the airway adapter 100, such as from the pressure case 110, is adapted to be placed around an ear of the human subject. As is more clearly seen in FIGS. 7 and 14, in the illustrated embodiment, the pressure sensing tube 120 is adapted to be placed around the left ear of the human subject. The embodiments are, though, not limited thereto. In another embodiment, the pressure sensing tube 120 extends from the opposite side wall 114 of the pressure case 110 to thereby be adapted to be placed around the right ear of the human subject. Placing the pressure sensing tube 120 around one ear of the human subject and, which is further discussed herein, the sensor cable 240 of the mainstream capnography sensor 200 around the other ear of the human subject will position the airway adapter 100 below the nostrils of the human subject to thereby enable collection of nasally exhaled air through the at least one nasal prong 131, 132.

In an embodiment, the pressure sensing tube 120 extends acutely from the pressure case 110. This is more clearly seen in FIGS. 1, 2, 11 and 12. Hence, in this embodiment, the pressure sensing tube 120 extends from the pressure case 110 directed towards the face of a human subject rather than extending orthogonally from one of the sides of the pressure case 110. Such an acute extension of the pressure sensing tube 120 facilitates routing of the pressure sensing tube 120 around one of the human subject's ear as the pressure sensing tube 120 is thereby extending and being directed towards the ear.

A significant advantage of the airway adapter 100 is that it only needs a single pressure sensing tube 120. Hence, in an embodiment, the airway adapter 100 comprises a single pressure sensing tube 120 and in particular no other tubes.

Hence, an embodiment of the present invention merely requires a single pressure sensing tube 120 and a single sensor cable 240 that are together used to attach the airway adapter 100 and the connected mainstream capnography sensor 200 on the face of the human subject by routing the pressure sensing tube 120 and the sensor cable 240 around respective ears.

The airway adapter 100 of the invention is thereby easy to use also for non-medical personnel, including the human subject itself, and attach on the face by having only a single tube 120 and a single cable 240 to handle.

The airway case 140 comprises at least one passage wall 144, 145, 146, 147 defining the airway passage 141. For instance, the airway passage 141 could comprise four connected passage walls 144, 145, 146, 147 defining and enclosing the airway passage 141. In such an embodiment, the airway passage 141 typically has a quadratic or rectangular cross-section. The embodiments are, though, not limited thereto. For instance, a single cylinder wall could be used to define an airway passage 141 having a circular cross-section.

In the embodiment shown in FIGS. 1-3, the airway case 140 comprises multiple, typically four, passage walls 144, 145, 146, 147 defining the airway passage 141. In such an embodiment, a first passage wall 144 of the multiple passage walls 144, 145, 146, 147 comprises the first light window 142 and a second passage wall 145 of the multiple passage walls 144, 145, 146, 147 comprises the second light window 143. In a preferred embodiment, the first and second passage walls 144, 145 are opposite passage walls 144, 145 facing each other. In such an embodiment, the at least one light source 220 and the at least one light detector 230 will be arranged opposite each other with the airway passage 141 running therebetween.

The first and second passage walls 144, 145 are preferably interconnected by a third passage wall 146 and a fourth passage wall 147. The fourth passage wall 147 is then facing the face of the human subject when the airway adapter 100 is attached on the face of the human subject and the third passage wall 146 is opposite to this fourth passage wall 147, i.e., faces away from the face in the embodiment shown in FIGS. 1-3. In the embodiment, shown in FIGS. 11-13, the first passage wall 144 faces the face of the human subject when the airway adapter 100 is attached on the face of the human subject and the second passage wall 145 is opposite to this first passage wall 144, i.e., faces away from the face. The first and second passage walls 144, 145 are preferably interconnected by a third passage wall 146 and a fourth passage wall 147.

The mainstream capnography sensor 200 is connectable to the airway adapter 100. Any type of connection or attachment solution that allows the mainstream capnography sensor 200 to be attached to the airway adapter 100 could be used. In an embodiment, the airway adapter 100 therefore comprises a connector 150 connectable to the mainstream capnography sensor 200 and arranged to position the mainstream capnography sensor 200 onto the airway adapter 100 to align the at least one light source 220 with the first light window 142 and align the at least one light detector 230 with the second light window 143, see FIGS. 1 and 4.

The connector 150 could, for instance, be attached to the above-mentioned third passage wall 146 of the multiple passage walls 144, 145, 146, 147 facing away from the face of the human subject when earing the airway adapter 100 as shown in FIGS. 1 and 4.

As an illustrative example, the connector 150 could provide a snap-fit connection of the mainstream capnography sensor 200 onto the airway adapter 100. The connector 150 could, for instance, comprise two opposite snap-fit connectors configured to grip the mainstream capnography sensor 200 when positioned between the opposite snap-fit connectors as shown in FIGS. 1 and 4. Also other connector solutions are possible. For instance, the mainstream capnography sensor 200 could comprise the snap-fit connectors, which are then configured to grip the airway case 140, see FIG. 3.

A further solution is to have a magnet attached to the third passage wall 146 of the airway case 140 with a matching magnet or iron piece in the mainstream capnography sensor 200 to magnetically attach the mainstream capnography sensor 200 to the airway adapter 100. In another embodiment, the mainstream capnography sensor 200 comprises a magnet with an iron piece in the airway adapter 100, such as attached to the third passage wall 146 of the airway case 140.

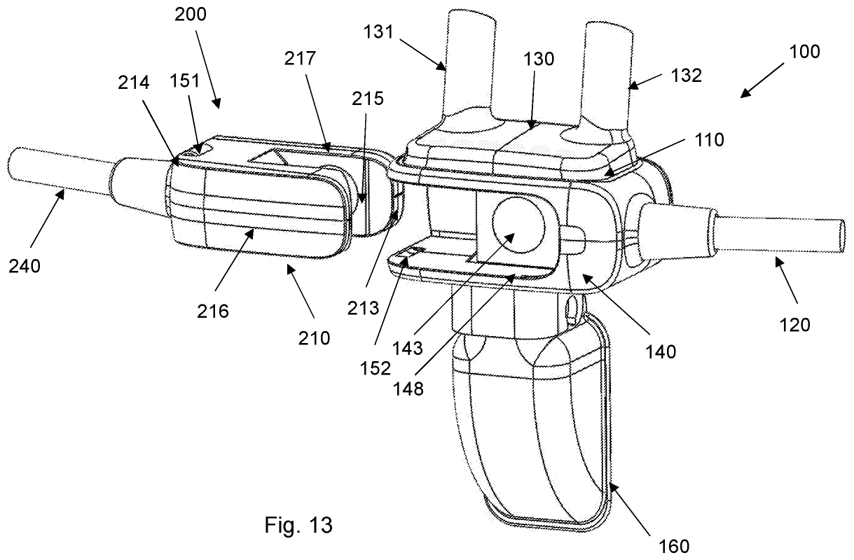

FIGS. 11 to 13 illustrate yet another alternative. In this embodiment, the mainstream capnography sensor 200 comprises a U-shaped sensor housing 210 as best seen in FIG. 13. The sensor housing 210 thereby comprises two legs or arms 216, 217, also referred to as protrusions or extensions herein, each comprising a respective light window 211, 212 and extending from a second short end 214 of the sensor housing 210 to a first short end 213 of the sensor housing 210. The sensor housing 210 thereby has an indentation defined by the extending legs or arms 216, 217. In such an embodiment, the airway case 140 preferably comprises receiving grooves 148 matching the legs or arms 216, 217 of the sensor housing 210 and, which may receive arms 216, 217 when the mainstream capnography sensor 200 is connected to the airway adapter 140 as shown in FIG. 13. In such an embodiment, the mainstream capnography sensor 200 could be releasably locked to the airway adapter 100 through a snap-fit connection. Such a snap-fit connection could then be in the form of a protrusion 151 arranged in the sensor housing 210, typically in connection with the second short end 214 and then a matching recess 152 in the airway case 140. When the legs or arms 216, 217 of the sensor housing 210 are fully inserted into the receiving grooves 148 of the airway case 140 the protrusion 151 enters the matching recess 152 and thereby connects the mainstream capnography sensor 200 to the airway adapter 100. In a typical embodiment, two protrusions 151 are arranged on opposite sides in the sensor housing 210 and the airway case 140 then comprises two matching recesses 152. In an alternative embodiment, the one or two matching recesses 152 are instead in the sensor housing 210 with the one or more protrusions 151 present in the airway case 140.

Thus, various mechanical and magnetically connection solutions are possible for the connector 150 of the airway adapter 100.

As is shown in FIG. 1, in an embodiment, the prong case 130 is arranged on the pressure case 110 to thereby be positioned above the pressure case 110 when the airway adapter 100 is attached on the face of the human subject. Correspondingly, in an embodiment, the pressure case 110 is arranged on the airway case 140 to thereby be positioned above the airway case 140 when the airway adapter 100 is attached on the face. FIG. 11 illustrates an alternative embodiment, in which the prong case 130 is arranged adjacent or next to the gas case 110. In fact, the prong case 130 and the gas case 110 could arranged as a common case or structure that could be detachable from the airway case 140.

The airway adapter 100 may be configured to not only collect nasally exhaled air through the at least one nasal prong 131, 132. In an embodiment, the airway adapter 100 may also comprise a gas guide 160 adapted to be disposed in front of a mouth of the human subject to collect mouth-exhaled breath from the human subject and guide the mouth-exhaled breath into the airway passage 141.

In an embodiment, this gas guide 160 is attached to and extending below the airway case 140. The side 161 of the gas guide 160 facing the mouth of the human subject when the airway adapter 100 is worn is preferably scoop shaped to collect the air exhaled from the mouth. The gas guide 160 also comprises a gas passage 162, such as in the form of a U-shape gas passage 162 that is in fluid communication with the air passage 141. This means that when the human subject exhales by the mouth, the exhaled air hits the side 161 of the gas guide 160 facing the mouth and is guided, by the scoop-shape of this side 161 towards the gas passage 162 and further into the air passage 141. In such an embodiment, both nasally exhaled air and mouth-exhaled air will pass through air passage 141.

The gas guide 160 is preferably detachably connected to the airway adapter 100, and in particular to the airway case 140, and extends below the airway case 140 when the airway adapter 100 is attached on the face of the human subject. Hence, in a preferred embodiment, the attachment 163 of the gas guide 160 to the airway case 140 is a releasable attachment 163 so that the gas guide 160 could be removed from the airway adapter 100 if the human subject would not like to use it to collect mouth-exhaled air but rather only collect nasally-exhaled air by the airway adapter 100.

The attachment 163 may also be a pivotal attachment so that the gas guide 160 is allowed to swing at least slightly relative to the airway case 140. This allows the gas guide 160 to be adjusted close to the mouth of the human subject.

The airway adapter 100 including the pressure sensing tube 120 is preferably a disposable airway adapter 100. In such a case, the airway adapter 100 could be used by a user and then disposed when no longer needed. This should be compared to the mainstream capnography sensor 200 and the portable monitor 40, to be further described herein, which are preferably reusable. This means that the mainstream capnography sensor 200 and the portable monitor 40 could then be used by different users but each such user then has his/her own disposable airway adapter 100. The user may also dispose and replace the disposable airway adapter 100 following use thereof for a given period of time.

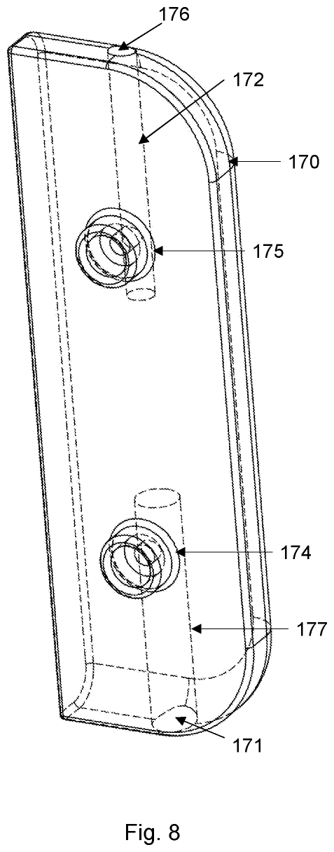

The present invention also relates to an airway adapter arrangement 10, see FIGS. 7 and 8. The airway adapter arrangement 10 comprises an airway adapter 100 according to the invention and a pressure measurement adapter 170 attached to the pressure sensing tube 120. The pressure measurement adapter 170 comprises a pressure sensing channel 172 in fluid communication with pressure sensing tube 120, a first pressure measuring port 175 in fluid communication with the pressure sensing channel 172 and a second pressure measuring port 174 in fluid communication with ambient air.

The pressure sensing tube 120 could be detachably attached to a first port 176 and could thereby be removed from the pressure measurement adapter 170. In another embodiment, the pressure sensing tube 120 is fixed to, i.e., permanently attached to, the pressure measurement adapter 170 at the first port 176 to fluidly connect the pressure sensing tube 120 with the pressure sensing channel 172. In such a case, the pressure sensing tube 120 could be glued or welded to the pressure measurement adapter 170.

The first port 176 is connected to the pressure sensing channel 172 and the pressure sensing channel 172 thereby extends at least between the first port 176 and the first pressure measuring port 175.

In an embodiment, the pressure measurement adapter 170 also comprises a pressure control channel 177 in fluid communication with the second pressure measuring port 174 and ambient air. For instance, the pressure control channel 177 could end at a second port 171 or opening in the pressure measurement adapter 170 that is thereby opens up to ambient air. The pressure control channel 177 then extends at least between this second port 171 and the second pressure measuring port 174.

In an embodiment, the airway adapter arrangement 10 is a disposable airway adapter arrangement 10. Hence, in such an embodiment, not only the airway adapter 100 but also the pressure measurement adapter 170 are disposable and could be discarded following use.

The airway adapter 100 and the pressure measurement adapter 170 of the airway adapter arrangement 10 are typically made of plastics, including various plastic materials.

The present invention also relates to a capnography arrangement 20, see FIGS. 4-7, 13. The capnography arrangement 20 comprises an airway adapter 100 according to the invention or an airway adapter arrangement 10 according to the invention. The capnography arrangement 20 also comprises a mainstream capnography sensor 200. The mainstream capnography sensor 200 comprises a sensor housing 210 comprising a first light window 211 and a second light window 212. The sensor housing 210 also comprises at least one light source 220 arranged in the sensor housing 210 to emit infrared (IR) light through the first light window 211 and at least one light detector 230 arranged in the sensor housing 210 to detect IR light through the second light window 212. The mainstream capnography sensor 200 further comprises a sensor cable 240 attached to the sensor housing 210 and in electrical communication with the at least one light source 220 and the at least one light detector 230.

The first light window 211 of the sensor housing 210 is aligned with the first light window 142 of the airway case 140 and the second light window 212 of the sensor housing 210 is aligned with the second light window 143 of the airway case 140 when the mainstream capnography sensor 200 is attached to the airway adapter 100.

The capnography system 20 thereby comprises the previously described and in FIGS. 1-3, 11-13 shown airway adapter 100, the mainstream capnography sensor 200 and optionally, but preferably also the pressure measurement adapter 170.

In an embodiment, the mainstream capnography sensor 200 is attached to the airway adapter 100 as shown in FIG. 7 or 13, such as using the previously described connector 150 of the airway adapter 100 or a connector of the mainstream capnography sensor 200. In this latter case, the mainstream capnography sensor comprises a connector connectable to the airway adapter 100 and arranged to position the mainstream capnography sensor 200 onto the airway adapter 100 to align the first light window 211 of the sensor housing 210 with the first light window 142 of the airway case 140 and align the second light window 212 of the sensor housing 210 with the second light window 143 of the airway adapter 100. In these embodiments, when the mainstream capnography sensor 200 is attached to the airway adapter 100, the first light windows 142, 211 of the airway case 140 and the sensor housing 210 are aligned to each other and the second light windows 143, 212 of the airway case 140 and the sensor housing 210 are likewise aligned to each other. This means that IR light from the at least one light source 220 is then directed through the first light window 211 of the sensor housing 210 and the first light window 142 of the airway case 140 and into the airway passage 141. The emitted IR light will flow through the second light window of the airway case 140 and the second light window 212 of the sensor housing 210 to be captured by the at least one light detector 230.

The light windows 211, 212 of the sensor housing 210 could be windows made of optically transparent material enabling the IR light from the light source 220 to pass through the light windows 211, 212. Illustrative, but non-limiting, examples of such materials include optically transparent plastics, glass and sapphire crystal.

In the embodiment shown in FIGS. 1-7, the light windows 142, 143, 212, 213 of the airway case 140 and of the mainstream capnography sensor 200 are arranged so that IR light emitted from the at least one light source 220 through the airway passage 141 and toward the at least one detector 230 is in a direction from one side of the airway adapter 100 towards the opposite of the airway adapter 100 when positioned at the face of a user. FIGS. 11-13 illustrate an alternative embodiment, in which the light windows 142, 143, 212, 213 of the airway case 140 and of the mainstream capnography sensor 200 are arranged so that IR light emitted from the at least one light source 220 is perpendicularly to the embodiment shown in FIGS. 1-7, i.e., in a direction towards or away from the face of the user then the airway adapter 100 is positioned at the face of a user.

In an embodiment, the pressure sensing tube 120 and the sensor cable 240 extend in opposite directions from the pressure case 110 and the sensor housing 210 respectively, when the mainstream capnography sensor 200 is connected to the airway adapter 100. Accordingly, the pressure sensing tube 120 and the sensor cable 240 are extended to enable one of the pressure sensing tube 120 and the sensor cable 240 to be routed around one of the human subject's ear with the other of the pressure sensing tube 120 and the sensor cable 240 around the other ear as shown in FIG. 14.

Hence, the pressure sensing tube 120 is adapted to be placed around one ear of the human subject and the sensor cable 240 is adapted to be placed around the other ear of the human subject, see FIG. 14.

The pressure case 110 comprises a first short end or side 113 and a second, opposite short end or side 114. The sensor housing 210 correspondingly comprises a first short end or side 213 and a second, opposite short end or side 214. The first short ends or sides 113, 213 of the pressure case 110 and the sensor housing 210 face the same direction and the second, opposite short ends or sides 114, 214 of the pressure case 110 and the sensor housing 210 face the same direction when the mainstream capnography sensor 200 is connected to the airway adapter 100. In such a case, the pressure sensing tube 120 extends, preferably acutely, from the first short end or side 113 of the pressure case 110 and the sensor cable 240 extends, preferably acutely, from the second short end or side 214 of the sensor housing 210.

The preferred acute extension of the pressure sensing tube 120 from the pressure case 110 and the preferred acute extension of the sensor cable 240 from the sensor housing 210 facilitate a routing of the pressure sensing tube 120 and the sensor cable 240 towards and over the outer ears of the human subject to thereby keep the airway adapter 100 with connected mainstream capnography sensor 200 positioned below the nostrils of the human subject.

As mentioned in the foregoing, the airway adapter 100 preferably comprises a single pressure sensing tube 120 to propagate the pressure wave or signal from the pressure case 110 to the pressure measurement adapter 170. Correspondingly, the mainstream capnography sensor 200 preferably comprises a single sensor cable 240 for providing power to the at least one light source 220 and the at least one light detector 230 and forward output signals from the at least one light detector 230 to a portable monitor 40.

In an embodiment, the sensor housing 210 comprises an indentation 215. In such an embodiment, the first and second light windows 211, 212 of the sensor housing 210 face the indentation 215. At least a portion of the airway case 140 is adapted to be positioned in the indentation 215 when the mainstream capnography sensor 200 is connected to the airway adapter 100.

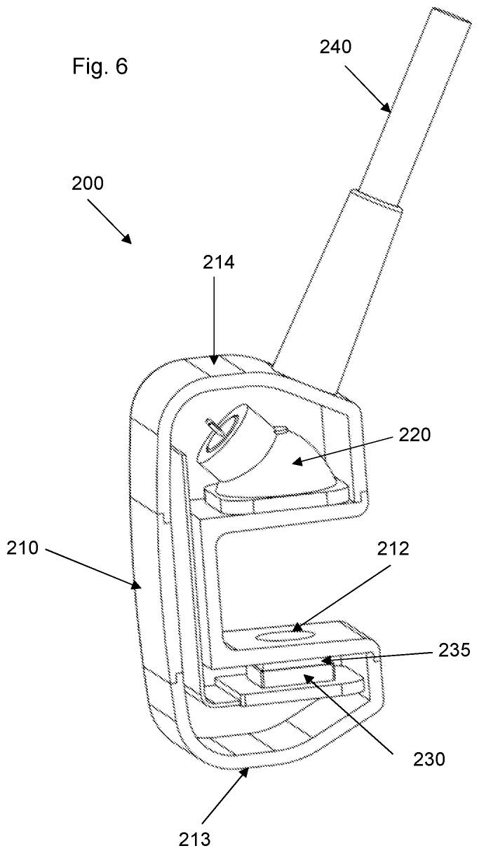

Hence, as shown in FIGS. 6 and 13, the sensor housing 210 of the mainstream capnography sensor 200 has a general U-shape with the two light windows 211, 212 facing the space between the two “arms” or “legs” 216, 217 of the U. In such a case, at least a portion of the airway case 140 is adapted to be positioned in this space so that a portion of the airway passage 141 will be present between the two “arms” or “legs” 216, 217 and the two light windows 211, 212 of the sensor housing 210 will face the intermediate airway passage 141.

In an embodiment, the sensor housing 210 comprises a single light source 220 configured to emit IR light. In another embodiment, the sensor housing 210 comprises multiple light sources 220 configured to emit IR light, typically of different wavelengths or wavelength intervals.

The light source 220 is preferably configured to emit IR light at one or more wavelengths within the spectrum from 3 μm up to 5 μm. In an embodiment, the light source 220 emits IR light of a single wavelength, such 4.3 μm, at multiple separate wavelengths, such at 3.9 μm and 4.3 μm, or at a band or spectrum of wavelengths, such as from 3 μm up to 5 μm.

In an embodiment, the sensor housing 210 comprises a single light detector 230 configured to detect IR light emitted from the at least one light source 220 and having passed through at least a portion of the airway passage 141. In another embodiment, the sensor housing 210 comprises multiple light detectors 230.

In an embodiment, the sensor housing 210 comprises a respective optical bandpass filter 235 interposed between the second light window 212 of the sensor housing 210 and the at least one light detector 230.

In an embodiment, the optical bandpass filter 235 has a center wavelength (CWL) of 4.3 μm and a full-width at half-maximum (FWHM) of about 100 nm. Such an optical bandpass filter 235 is suitable for measurement of CO₂ by the light detector 230.

In another embodiment, the sensor housing 210 of the mainstream capnography sensor 200 comprises the above-mentioned optical bandpass filter 235 (CWL=4.3 μm, FWHM ~100 nm) and a light detector 230 for detection and measurement of CO₂ and a reference light detector 230 and a reference optical bandpass filter for reference light measurements. The reference light detector 230 and reference optical bandpass filter can then be used to compensate for, for instance, drift in the light source 220, moisture or dirt on the light windows 142, 143, 211, 212, etc. In an embodiment, the reference bandpass filter has a CWL of 3.9 μm and a FWHM of about 100 nm.

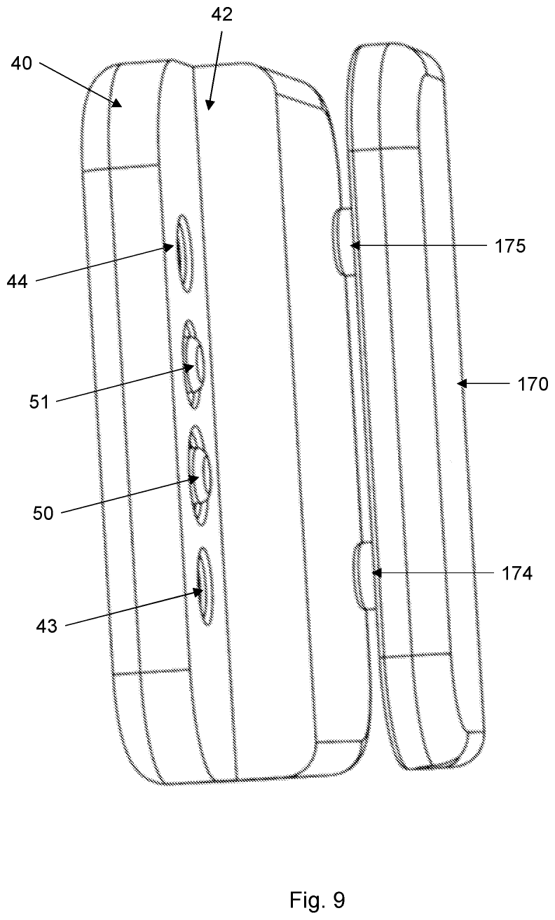

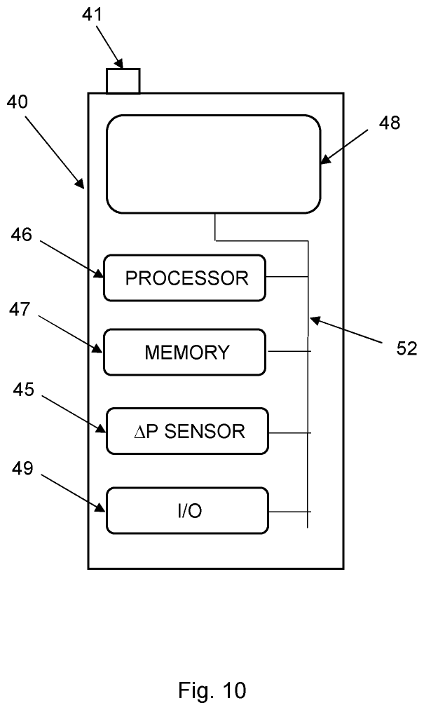

A further aspect of the invention relates to a somnography system 1, see Figs. FIGS. 7, 9-10. The somnography system 1 comprises the capnography arrangement 20 of the invention and in particular such a capnography arrangement 20 comprising an airway adapter arrangement 10 according to the invention. The somnography system 1 also comprises a portable monitor 40 for monitoring a spontaneously breathing human subject. The portable monitor 40 comprises a sensor port 41 connectable to a sensor cable 240 of the mainstream capnography sensor 200. The portable monitor 40 also comprises an adapter receptacle 42 connectable to the pressure measurement adapter 170. A first pressure port 44 of the portable monitor 40 is configured to be in fluid communication with the first pressure measuring port 175 of the pressure measurement adapter 170 and a second pressure port 43 of the portable monitor 40 is configured to be in fluid communication with the second pressure measuring port 174 of the pressure measurement adapter 170. The portable monitor 40 also comprises a differential pressure sensor 45 in fluid communication with the first pressure port 44 and the second pressure port 43. The differential pressure sensor 45 is configured to measure a pressure difference between the first pressure port 44 and the second pressure port 43 and generate an output signal representative of the pressure difference.

The portable monitor 40 also comprises a processor 46 communicatively connected to the sensor port 41 and the differential pressure sensor 45 and a memory 47 coupled to the processor 46. The memory 47 comprises instructions executable by the processor 46 to cause the processor 46 to process an output signal received at the sensor port 41 and generated by the mainstream capnography sensor 200 to generate a CO₂ parameter value representative of partial pressure of CO₂ in the exhaled air from the human subject. The processor 46 is also caused to process the output signal from the differential pressure sensor 45 to generate a respiration signal.

The portable monitor 40 is thereby a portable device, i.e., handheld or more preferably hanging attached to the pressure sensing tube 120 and the sensor cable 240 as shown in FIG. 7, which can be used together with the mainstream capnography sensor 200 to monitor CO₂ partial pressure in the air exhaled by the human subject, i.e., the user, at the same time as the portable monitor 40 is able to monitor the respiration of the human subject.

The portable monitor 40 can thereby be used as a diagnostic tool for patient monitoring and in particular when there is a need to monitor partial pressure of CO₂ at the same time as monitoring respiration of the human subject, such as in connection with sleep study.

In an embodiment, the memory 47 comprises instructions executable by the processor 46 to cause the processor 46 to process the output signal received at the sensor port 41 and generated by the mainstream capnography sensor 200 to generate an end-tidal CO₂ (ETCO₂) value.

EtCO₂ monitoring is a non-invasive technique that measures the partial pressure of maximum concentration of CO₂ at the end of an exhaled breath. EtCO₂ is typically expressed as a percentage of CO₂ or mmHg. Normal values for EtCO₂ are 5 to 6 % CO₂, which corresponds to 35-45 mmHg at sea level.

The processor 46 is typically configured to identify a local maximum in the CO₂ waveform or signal corresponding to the end of an expiration. This local maximum is followed by a sharp drop in the CO₂ waveform or signal corresponding to an inspiration. The local maximum in the CO₂ waveform or signal corresponds to the EtCO₂.

Generally, airflow and respiratory effort data are utilized during standard PSG to monitor respiration and detect the presence of apneas, hypopneas, respiratory effort related arousals (RERAs) and other sleep related breathing events. Elevated EtCO₂ readings may be associated with hypoventilation, whereas low values may be associated with hyperventilation or anxiety.

The somonography system 1 of the invention thereby enables simultaneous monitoring and recording of EtCO₂ (partial pressure of CO₂ in exhaled air) and respiratory effort (respiration signal).

In an embodiment, the memory 47 comprises instructions executable by the processor 46 to cause the processor 46 to determine a trend in the EtCO₂ value during a measurement period.

In an embodiment, the memory 47 comprises an EtCO₂ high threshold value and/or an EtCO₂ low threshold value. In this embodiment, the memory 47 comprises instructions executable by the processor 46 to cause the processor 46 to compare the EtCO₂ value with the EtCO₂ high threshold value and/or the EtCO₂ low threshold value. In this embodiment, the processor 46 is also caused to generate an EtCO₂ high alarm signal if the EtCO₂ value exceeds the EtCO₂ high threshold value or an EtCO₂ low alarm signal if the EtCO₂ value is below the EtCO₂ low threshold.

Illustrative, but non-limiting, examples of the EtCO₂ high and low threshold values include 50 mmHg and 0-30 mmHg.

In an embodiment, the memory 47 comprises instructions executable by the processor 46 to cause the processor 46 to generate a no breaths alarm if no valid breath is detected in the respiration signal during a defined measurement period, such as 20 s.

In an embodiment, the portable monitor 40 also comprises a display screen 48 communicatively connected to the processor 46. In this embodiment, the memory 47 comprises instructions executable by the processor 46 to cause the processor 46 to display the CO₂ parameter value and/or waveform on the display screen 48 and display the respiration signal on the display screen 48.

The processor 46 may be also caused to display the above-described ETCO₂ value.

The display screen 48 could display the one or more diagnostic parameters as single value and/or as a trend, such as a graph showing how the values of the one or more diagnostic parameters change over time.

The above-described alarms could be visible alarms presented on the display screen 48 and/or audible alarms.

In an embodiment, the portable monitor 40 comprises a communication unit 49 communicatively connected to the processor 46. In such an embodiment, the memory 47 comprises instructions executable by the processor 46 to cause the processor 46 to instruct the communication unit 49 to transmit the CO₂ parameter value or information representative of the CO₂ parameter value to an external device and instruct the communication unit 49 to transmit the respiration signal or information representative of the respiration signal to the external device.

The processor 46 may also cause the communication unit 49 to transmit the above-described ETCO₂ value to the external device.

The communication unit 49 may be a transmitter or transceiver configured to conduct wireless communication with the external device. In another embodiment, the communication unit 49 is an output port or a combined input and output (I/O) port for wired communication with the external device.

The external device could be a mobile phone, table or computer of the human subject or a computer or computer system of a physician or healthcare facility as illustrative but non-limiting examples.

The adapter receptacle 42 of the portable monitor 40 is connectable to the pressure measurement adapter 170. In an embodiment, a switch 50 of the portable monitor 40 is arranged in connection with the adapter receptacle 42 to indicate when the pressure measurement adapter 170 is connected to the adapter receptacle 42. In such an embodiment, the processor 46 is responsive to the output signal from the switch 50 and will then process the output signal from the differential pressure sensor 45 if, and preferably only if, the switch 50 indicates that the pressure measurement adapter 170 is connected to the adapter receptacle 42 of the portable monitor 40. In such a case, the differential pressure measurements will only take place when the portable monitor 40 is connected to the pressure measurement adapter 170 and the pressure sensing channel 172 of the pressure measurement adapter 170.

In an embodiment, the portable monitor 40 comprises a second switch 51. In such an embodiment, this second switch 51 could generate an output signal when an adapter 170 is connected to the adapter receptacle 42. The output signal from the second switch 51 is then indicative of the type of adapter 170 that is connected to the adapter receptacle 42. For instance, the adapter receptacle 42 of the portable monitor 40 could be configured to be connected to different types of adapters 170. As an example, a first pressure measurement adapter 170 is to be used in connection with adult users and a second pressure measurement adapter 170 is to be used in connection with children. Another example is to have the option of connect either a pressure measurement adapter 170 or an adapter for gas flow measurements to the adapter receptacle 42. In this latter case, the adapter for gas flow measurements could be connected to a gas source, such as an oxygen source, for measurement of a flow rate of oxygen delivery to a user.

The output signal from the second switch 51 can then be processed by the processor 46 to determine which particular adapter 170 that is connected to the adapter receptacle 42 and can thereby process the output signal from the differential pressure sensor 45 at least partly based on the type of adapter 170 that is currently connected to the adapter receptacle 42 as indicated by the output signal from the second switch 51.

The first pressure port 44 of the portable monitor 40 is in fluid communication with the first pressure measuring port 175 of the pressure measurement adapter 170 and the second pressure port 43 of the portable monitor 40 is in fluid communication with the second pressure measuring port 174 of the pressure measurement adapter 170 when the pressure measurement adapter 170 is connected to the adapter receptacle 42. In such a case, the differential pressure sensor 45 is able to measure a pressure difference between the first and second pressure ports 44, 43 and thereby between the first and second pressure measuring ports 175, 174 and between the pressure in the pressure sensing tube 120 and ambient pressure.

The processor 46 and the memory 47 are communicatively connected to each other. For instance, the portable monitor 40 could comprise a communication bus 52 allowing communication and data transfer between the processor 46 and the memory 47. Also the differential pressure sensor 45, the communication unit 49 and the display screen 48 could be connected to this communication bus 52.

The term processor should be interpreted in a general sense as any circuitry, system or device capable of executing program code or computer program instructions to perform a particular processing, determining or computing task. The processor 46 does not have to be dedicated to only execute the above-described steps, functions, procedure and/or blocks, but may also execute other tasks.

During use of the somnography system 1, the pressure measurement adapter 170 is attached to the adapter receptable 42 of the portable monitor 40, the mainstream capnography sensor 200 is connected to the airway adapter 100 and the sensor cable 240 is connected to the sensor port 41 of the portable monitor 40.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1-26. (canceled)

27. An airway adapter adapted to be disposed below nostrils of a human subject and comprising:
a prong case comprising at least one nasal prong arranged to collect nasally exhaled breath from the human subject;
a pressure case in fluid communication with the prong case via a pressure channel;
a pressure sensing tube in fluid communication with the pressure case; and
an airway case defining an airway passage in fluid communication with the prong case via a flow restriction, wherein the airway case comprises:
a first light window into the airway passage; and
a second light window into the airway passage, wherein the airway adapter is connectable to a mainstream capnography sensor comprising at least one light source and at least one light detector to position the mainstream capnography sensor onto the airway adapter to align the at last one light source with the first light window and align the at least one light detector with the second light window.

28. The airway adapter according to claim 27, wherein the pressure sensing tube is adapted to be placed around an ear of the human subject.

29. The airway adapter according to claim 27, wherein the pressure sensing tube extends acutely from the pressure case.

30. The airway adapter according to claim 27, wherein with the prong case comprises a first nasal prong and a second nasal prong arranged to collect nasally exhaled breath from the human subject.

31. The airway adapter according to claim 27, wherein
the airway case comprises multiple passage walls defining the airway passage;
a first passage wall of the multiple passage walls comprises the first light window; and
a second, opposite passage wall of the multiple passage walls comprises the second light window.

32. The airway adapter according to claim 27, further comprising a connector connectable to the mainstream capnography sensor and arranged to position the mainstream capnography sensor onto the airway adapter to align the at last one light source with the first light window and align the at least one light detector with the second light window.

33. The airway adapter according to claim 27, further comprising receiving grooves adapted to receive matching arms of the mainstream capnography sensor to position the mainstream capnography sensor onto the airway adapter to align the at last one light source arranged in one matching arm with the first light window and align the at least one light detector arranged in another matching arm with the second light window.

34. The airway adapter according to claim 27, further comprising a gas guide adapted to be disposed in front of a mouth of the human subject to collect mouth-exhaled breath from the human subject and guide the mouth-exhale breath into the airway passage.

35. The airway adapter according to claim 34, wherein the gas guide is detachably attached to the airway case and extends below the airway case when the airway adapter is attached on the face of the human subject.

36. The airway adapter according to claim 27, wherein the airway adapter is a disposable airway adapter.

37. An airway adapter arrangement comprising:
an airway adapter according to claim 27; and
a pressure measurement adapter attached to the pressure sensing tube and comprising:
a pressure sensing channel in fluid communication with the pressure sensing tube;
a first pressure measuring port in fluid communication with the pressure sensing channel; and
a second pressure measuring port in fluid communication with ambient air.

38. The airway adapter arrangement according to claim 37, wherein the airway adapter arrangement is a disposable airway adapter arrangement.

39. A capnography arrangement comprising:
an airway adapter according to claim 27;
a mainstream capnography sensor comprising:
a sensor housing comprising:
a first light window;
a second light window;
at least one light source arranged in the sensor housing to emit infrared (IR) light through the first light window; and
at least one light detector arranged in the sensor housing to detect IR light through the second light window; and
a sensor cable attached to the sensor housing and in electrical communication with the at least one light source and the at least one light detector, wherein
the first light window of the sensor housing is aligned with the first light window of the airway case and the second light window of the sensor housing is aligned with the second light window of the airway case when the mainstream capnography sensor is connected to the airway adapter.

40. The capnography arrangement according to claim 39, wherein the pressure sensing tube and the sensor cable extends in opposite directions from the pressure case and the sensor housing, respectively, when the mainstream capnography sensor is connected to the airway adapter.

41. The capnography arrangement according to claim 39, wherein
the pressure sensing tube is adapted to be placed around one ear of the human subject; and
the sensor cable is adapted to be placed around the other ear of the human subject.

42. The capnography arrangement according to claim 39, wherein
the pressure case comprises a first short end and a second, opposite short end;
the sensor housing comprises a first short end and a second, opposite short end;
the pressure sensing tube extends acutely from the first short end of the pressure case;
the sensor cable extends acutely from the second, opposite short end of the sensor housing; and
the first short ends of the pressure case and the sensor housing face the same direction and the second, opposite short ends of the pressure case and the sensor housing face the same direction when the mainstream capnography sensor is connected to the airway adapter.

43. The capnography arrangement according to claim 39, wherein
the sensor housing comprises an indentation;
the first and second light windows of the sensor housing face the indentation; and
at least a portion of the airway case is adapted to be positioned in the indentation when the mainstream capnography sensor is connected to the airway adapter.

44. The capnography arrangement according to claim 39, wherein the sensor housing comprises a respective optical bandpass filter interposed between the second light window of the sensor housing and the at least one light detector.

45. The capnography arrangement according to claim 39, wherein the mainstream capnography sensor comprises a connector connectable to the airway adapter and arranged to position the mainstream capnography sensor onto the airway adapter to align the first light window of the sensor housing with the first light window of the airway case and align the second light window of the sensor housing with the second light window of the airway case.

46. The capnography arrangement according to claim 39, wherein
the sensor housing is a U-shaped sensor housing comprising two arms;
the first light window and the at least one light source are arranged in a first arm of the two arms;
the second light window and the at least one light detector are arranged in a second arm of the two arms; and
the airway case comprises receiving grooves arranged to receive the two arms to position the mainstream capnography sensor onto the airway adapter to align the first light window of the sensor housing with the first light window of the airway case and align the second light window of the sensor housing with the second light window of the airway case.

47. A somnography system comprising:
a capnography arrangement according to claim 39 and further comprising:
a pressure measurement adapter attached to the pressure sensing tube and comprising:
a pressure sensing channel in fluid communication with the pressure sensing tube;
a first pressure measuring port in fluid communication with the pressure sensing channel; and
a second pressure measuring port in fluid communication with ambient air; and
a portable monitor for monitoring a spontaneously breathing human subject, wherein the portable monitor comprises:
a sensor port connectable to the sensor cable of the mainstream capnography sensor;
an adapter receptacle connectable to the pressure measurement adapter;
a first pressure port configured to be in fluid communication with the first pressure measuring port of the pressure measurement adapter;
a second pressure port configured to be in fluid communication with the second pressure measuring port of the pressure measurement adapter;
a differential pressure sensor in fluid communication with the first pressure port and the second pressure port and configured to measure a pressure difference between the first pressure port and the second pressure port and generate an output signal representative of the pressure difference;
a processor communicatively connected to the sensor port and the differential pressure sensor; and
a memory coupled to the processor and comprising instructions executable by the processor to cause the processor to:
process an output signal received at the sensor port and generated by the mainstream capnography sensor to generate a CO₂ parameter value representative of partial pressure of CO₂ in the exhaled air from the human subject; and
process the output signal from the differential pressure sensor to generate a respiration signal.

48. The somnography system according to claim 47, wherein the memory comprises instructions executable by the processor to cause the processor to process the output signal received at the sensor port and generated by the mainstream capnography sensor to generate an end-tidal CO₂ (ETCO₂) value.

49. The somnography system according to claim 47, wherein the portable monitor comprises a display screen communicatively connected to the processor, wherein the memory comprises instructions executable by the processor to cause the processor to:
display the CO₂ parameter value on the display screen; and
display the respiration signal on the display screen.

50. The somnography system according to claim 47, wherein the portable monitor comprises a communication unit communicatively connected to the processor, wherein the memory comprises instructions executable by the processor to cause the processor to:
instruct the communication unit to transmit the CO₂ parameter value or information representative of the CO₂ parameter value to an external device; and
instruct the communication unit to transmit the respiration signal or information representative of the respiration signal to the external device.

51. The somnography system according to claim 47, wherein the portable monitor comprises a switch arranged in connection with the adapter receptacle and configured to generate an output signal when the pressure measurement adapter is connected to the adapter receptacle, wherein the processor is communicatively connected to the switch and is configured to process the output signal from the differential pressure sensor if the output signal from the switch indicates that the pressure measurement adapter is connected to the adapter receptacle.

52. The somnography system according to claim 47, wherein the pressure measurement adapter is attached to the adapter receptable.
